(19) United States
(12) Patent Application Publication
(10) Pub. No.: US 2021/0219512 A1
(43) Pub. Date: Jul. 22, 2021

(54) CANNABIS PLANT NAMED 'OT-1'

(71) Applicant: Matthew Gordon, Oak Harbor, WA (US)

(72) Inventor: Matthew Gordon, Oak Harbor, WA (US)

(21) Appl. No.: 17/119,215

(22) Filed: Dec. 11, 2020

Related U.S. Application Data
(60) Provisional application No. 62/947,516, filed on Dec. 12, 2019.

(51) Int. Cl. A01H 6/28; A01H 5/12

(52) U.S. Cl. CPC A01H 6/28; A01H 5/12

(57) ABSTRACT

The unique Cannabis plant variety C. sativa ‘OT-1’ is provided.

### CROSS REFERENCE TO RELATED APPLICATIONS

This application claims the benefit of U.S. Provisional Application No. 62/947,516, entitled, “CANNABIS PLANT NAMED ‘OT-1’”, filed on Dec. 12, 2019. The entire content of the foregoing is hereby incorporated by reference in its entirety.

### LATIN NAME OF THE GENUS AND SPECIES

Genus—Cannabis.

Species—sativa.

### VARIETY DENOMINATION

The new Cannabis plant claimed is of the variety denominated ‘OT-1’.

### BACKGROUND OF THE INVENTION

### Field of the Invention

The present invention relates to a new and distinct annual variety of C. sativa hybrid, which has been given the variety denomination of ‘OT-1’. ‘OT-1’ is intended for use as medicinal herb for sale in cannabis dispensaries and as a home garden plant.

### Background of the Related Art

The genus Cannabis has been in use by humans for millennia, due to the multiplicity of its benefits to humans, including the considerable value and utility of its fiber, the nutritional value of its seeds, and the medicinal value of its floral parts and products made from them. Currently the genus is under intense legal commercialization in the United States as industrial hemp for a variety of purposes including biodegradable plastics and building materials, clothing, paper, food, fuel and medicines.

Cannabidiol (CBD) extracted from Cannabis is widely used in over-the-counter medicines and topical treatments and is also the active ingredient in the FDA-approved drug Epidiolex. CBD is just one of at least dozens—perhaps hundreds—of cannabinoids endogenous to Cannabis, tetrahydrocannabinol (THC) being the other cannabinoid that is most well-known. The cannabinoids as a group interact with the human endocannabinoid receptors, which are distributed in the brain and throughout the body. The study of the endocannabinoid system (ECS) in humans and other mammals is an area of increasing interest and holds tremendous promise for the future of medicine. See, e.g., Russo (2019). Cannabis and Pain, Pain Medicine, 20(10): 1093/pm/pnz227; and Russo (2016). Clinical Endocannabinoid Deficiency Reconsidered: Current Research Supports the Theory in Migraine, Fibromyalgia, Irritable Bowel, and Other Treatment-Resistant Syndromes, Cannabis Cannabinoid Res. 1(1): 154-165.

Non-hemp forms of Cannabis, frequently referred to as marijuana, have been legalized for medicinal use in many states and also for recreational use (sometimes called “adult use”) in a growing number of states including Alaska, Arizona, California, Colorado, Illinois, Maine, Massachusetts, Michigan, Montana, New Jersey, Nevada, Oregon, Vermont, and Washington. It is expected that the wave of legalization will continue to the point of some form of federal legalization or decriminalization.

Typically, marijuana products are available to users for purchase in specialized “dispensaries” that offer dried flower, edibles, tinctures, extracts, and the like. In some cases, a unique or unusual chemical profile, or chemotype, is attractive not only for flower sales but also for use in the preparation of extracts and/or isolates and for the manufacture of a variety of products that possess characteristics of the chemotype.

### SUMMARY OF THE INVENTION

Plants of the new cultivar were developed to remove negative effects such as fatigue, increased appetite, anxiety, memory loss, etc., that are experienced by a consumer of the plant.

‘OT-1’ originated in Burlington Wash., in 2005. The parents are of an unknown strain derived from “bagseed” found in flower purchased for consumption. It is believed to be related to ‘Mendo Purps’.

The variety was originally grown from a seed and then cloned. The clones were self-crossed for 13 generations, and offspring of those crosses were selected for efficacy in ameliorating memory loss. Several sibling crosses within a generation were conducted using the same selection criteria, seeking to combine the favorable effects of each. Each generation produced approximately 50-150 plants which were screened. At certain stages, when a combination of desired traits was achieved in a given plant, such plant was backcrossed to a parental or grandparental generation to obtain higher expression.

The variety was propagated asexually and with seeds between 2012 and 2018 and showed a uniformity in the population.

### BRIEF DESCRIPTION OF PICTURES

The accompanying photographs show the typical appearance of the new variety ‘OT-1’. The colors are as nearly true as is reasonably possible in a color representation of this type. Colors in the photographs may differ slightly from the color values cited in the detailed botanical description which accurately describes the colors of the new plant.

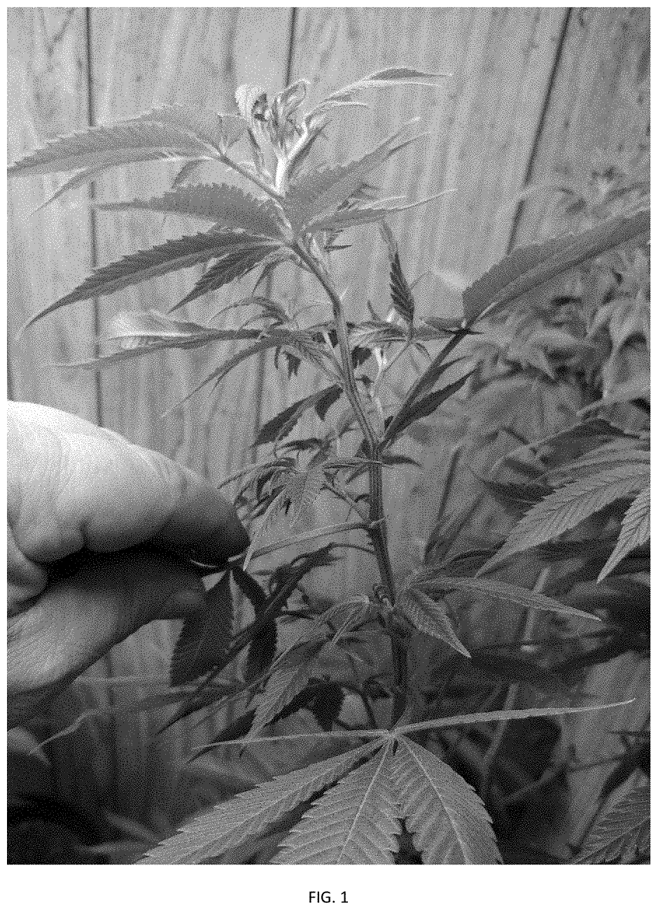

FIG. 1 is photograph of the plant at 6 weeks of age.

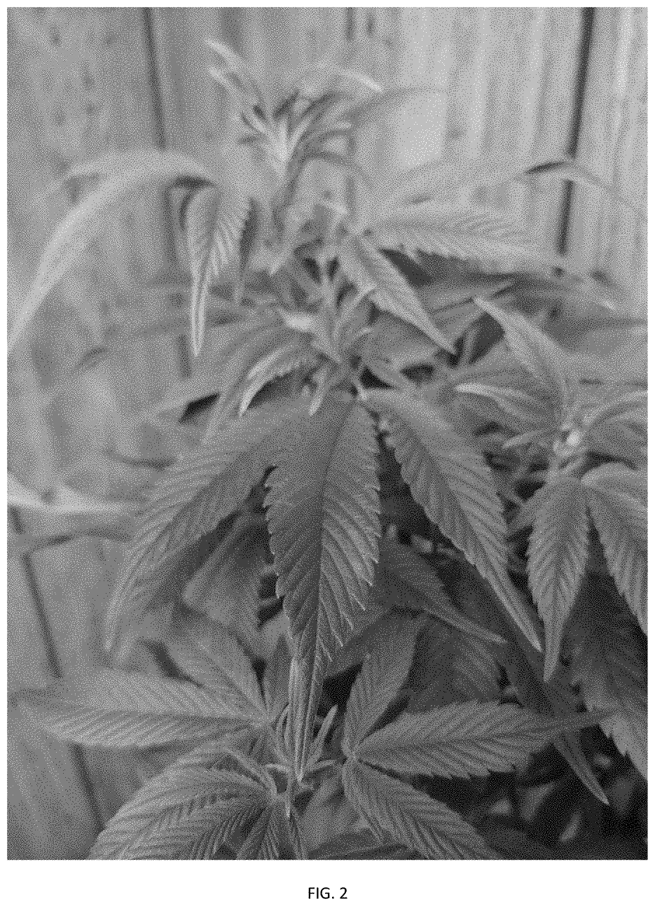

FIG. 2 is photograph of the plant at 6 weeks of age.

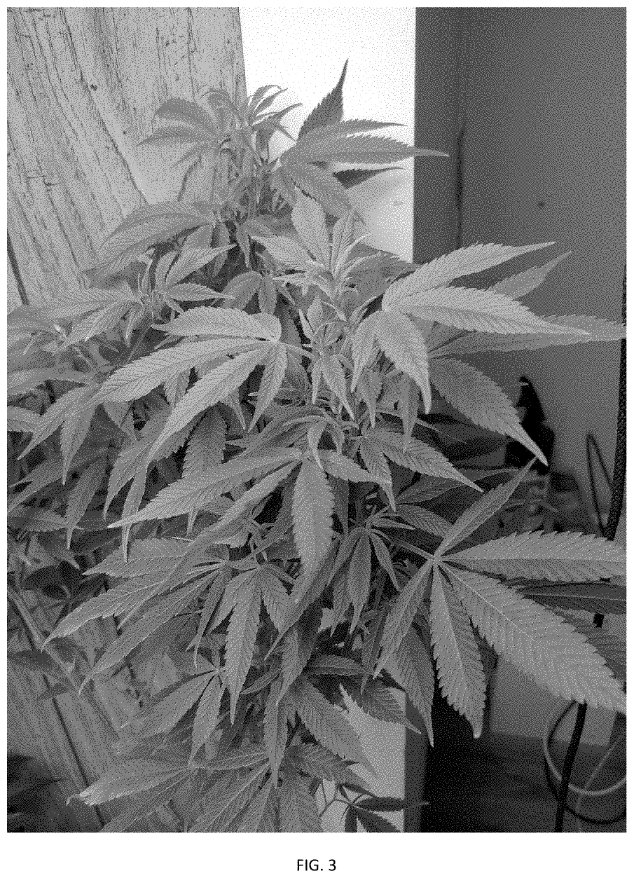

FIG. 3 is photograph of the plant at 6 weeks of age.

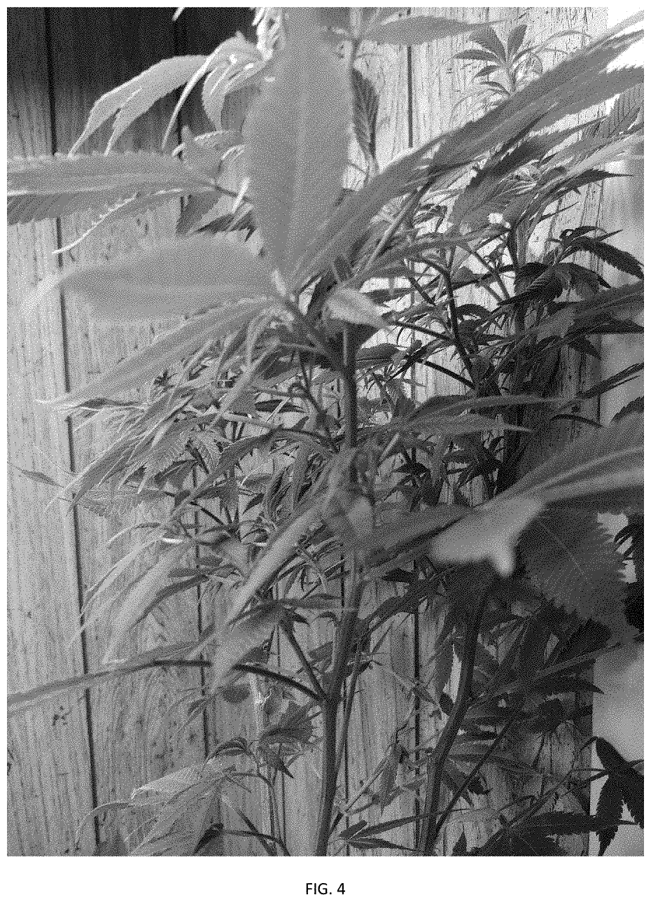

FIG. 4 is photograph of the plant at 6 weeks of age.

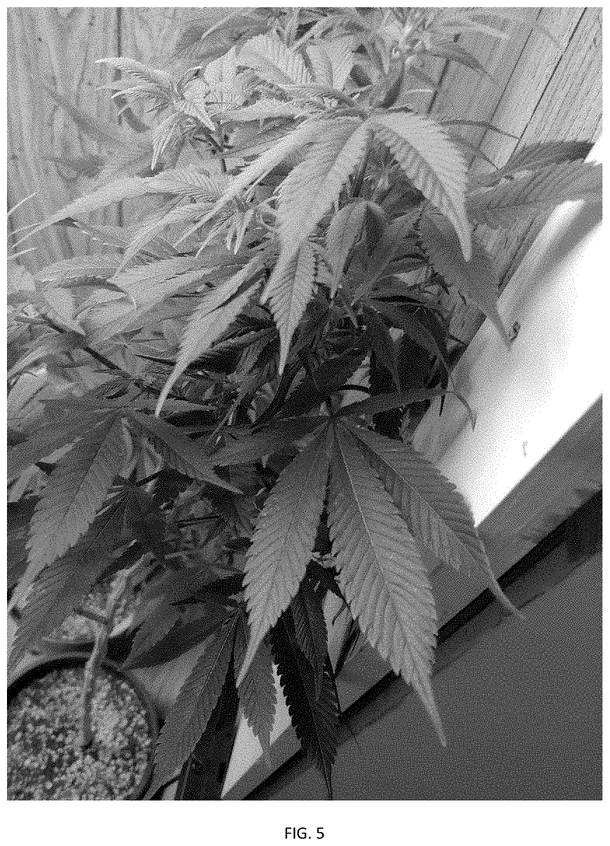

FIG. 5 is photograph of the plant at 6 weeks of age.

### DETAILED DESCRIPTION

Some embodiments of the invention relate to a seed from a Cannabis plant designated ‘OT-1’ wherein a representative sample of seed of said plant has been deposited under ______.

Some embodiments of the invention relate to a Cannabis plant, or plant part, tissue, or cell thereof produced by growing the seed of ‘OT-1’, or a descendant thereof. Plant parts can include the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like.

The plants, or plant parts of the invention can display a cannabinoid profile within the ranges set forth in Table 1, as defined herein. The productivity of any given cannabinoid and/or the amounts or ratios of cannabinoids, terpenes, and other plant products can be, by nature, quite variable. The variability can be contributed to by weather, latitude, soil and feeding conditions, pathogens, and numerous other agronomic, horticultural, and biological factors.

Some embodiments of the invention relate to methods of using the plant in a breeding program to produce cannabis progeny including a cannabinoid profile generally within the ranges as set forth in Table 1. Details of existing Cannabis plant varieties and breeding are described in Potter et al. (2011, World Wide Weed: Global Trends in Cannabis Cultivation and Its Control); Holland (2010, The Pot Book: A Complete Guide to Cannabis, Inner Traditions/Bear & Co, ISBN1594778981, 9781594 778988); Green I (2009, The Cannabis Grow Bible: The Definitive Guide to Growing Marijuana for Recreational and Medical Use, Green Candy Press, 2009, ISBN 1931160589, 9781931160582); Green II (2005, The Cannabis Breeder's Bible: The Definitive Guide to Marijuana Genetics, Cannabis Botany and Creating Strains for the Seed Market, Green Candy Press, 1931160279, 9781931160278); Starks (1990, Marijuana Chemistry Genetics, Processing & Potency, ISBN 0914171399, 9780914171393); Clarke (1981, Marijuana Botany, an Advanced Study: The Propagation and Breeding of Distinctive Cannabis, Ronin Publishing, ISBN 091417178X, 9780914171782); Short (2004, Cultivating Exceptional Cannabis: An Expert Breeder Shares His Secrets, ISBN 1936807122, 9781936807123); Cervantes (2004, Marijuana Horticulture: The Indoor/Outdoor Medical Grower's Bible, Van Patten Publishing, ISBN 187882323X, 9781878823236); Franck et al. (1990, Marijuana Grower's Guide, Red Eye Press, ISBN 0929349016, 9780929349015); Grotenhermen and Russo (2002, Cannabis and Cannabinoids: Pharmacology, Toxicology, and Therapeutic Potential, Psychology Press, ISBN 0789015080, 9780789015082); Rosenthal (2007, The Big Book of Buds: More Marijuana Varieties from the World's Great Seed Breeders, ISBN 1936807068, 9781936807062); Clarke, RC (Cannabis: Evolution and Ethnobotany 2013); King, J (Cannabible Vols 1-3, 2001-2006); and four volumes of Rosenthal's Big Book of Buds series (2001, 2004, 2007, and 2011), each of which is herein incorporated by reference in its entirety for all purposes.

The present invention also relates to variants, mutants and minor modifications of the seeds, plant parts and/or whole plants of the Cannabis plants of the present invention. Variants, mutants and minor modifications of the seeds, plants, plant parts, plant cells of the present invention can be generated by methods well known and available to one skilled in the art, including but not limited to, mutagenesis (e.g., chemical mutagenesis, radiation mutagenesis, transposon mutagenesis, insertional mutagenesis, signature tagged mutagenesis, site-directed mutagenesis, and natural mutagenesis), knock-outs/knock-ins, antisense and RNA interference. For more information of mutagenesis in plants, such as agents, protocols, see Acquaah et al. (Principles of plant genetics and breeding, Wiley-Blackwell, 2007, ISBN 1405136464, 9781405136464,) which is herein incorporated by reference in its entirety. Other kinds of modifications practiced in the Cannabis industry, including but not limited to feminization of seeds and/or day-length neutrality/autoflowering are also within the scope of the invention and are within the level of skill in the art to execute.

The present invention also relates to a mutagenized population of the Cannabis plants of the present invention, and methods of using such populations. In some embodiments, the mutagenized population can be used in screening for new Cannabis lines which comprises one or more or all of the morphological, physiological, biological, and/or chemical characteristics of Cannabis plants of the present invention.

In some embodiments, the new Cannabis plants obtained from the screening process comprise one or more or all of the morphological, physiological, biological, and/or chemical characteristics of Cannabis plants of the present invention, and one or more additional or different new morphological, physiological, biological, and/or chemical characteristic.

The present invention also provides any compositions or any products made from or isolated from the plants of the present invention. In some embodiments, the compositions/products comprise an extract of the plants. In some embodiments, the extract can contain a higher percentage of terpenes/terpenoids compared to extract isolated from a control Cannabis plant variety (e.g., an existing variety, such as a recreational Cannabis plant variety). In some embodiments, the invention relates to a smokable or edible product comprising the Cannabis plant, or plant part, tissue, cell, extract, or isolate.

The present invention provides methods of using the Cannabis plants or any parts, any compositions, or any chemicals derived from said plants of the present invention.

In some embodiments, the plants of the present invention can be used to produce new plant varieties. In some embodiments, the plants are used to develop new varieties or hybrids with desired phenotypes or genotypes.

In some embodiments, selection methods, e.g., molecular marker assisted selection, can be combined with breeding methods to accelerate the process. Additional breeding methods known to those of ordinary skill in the art include, e.g., methods discussed in Chahal and Gosal (Principles and procedures of plant breeding: biotechnological and conventional approaches, CRC Press, 2002, ISBN 084931321X, 9780849313219); Taji et al. (In vitro plant breeding, Routledge, 2002, ISBN 156022908X, 9781560229087); Richards (Plant breeding systems, Taylor & Francis US, 1997, ISBN 0412574500, 9780412574504); Hayes (Methods of Plant Breeding, Publisher: READ BOOKS, 2007, ISBN1406737062, 9781406737066); each of which is incorporated by reference in its entirety. The Cannabis genome has been sequenced (Bakel et al., The draft genome and transcriptome of Cannabis sativa, Genome Biology, 12(10):R102, 2011). Molecular makers for Cannabis plants are described in Datwyler et al. (Genetic variation in hemp and marijuana (Cannabis sativa L.) according to amplified fragment length polymorphisms, J Forensic Sci. 2006 March; 51(2):371-5.); Pinarkara et al., (RAPD analysis of seized marijuana (Cannabis sativa L.) in Turkey, Electronic Journal of Biotechnology, 12(1), 2009), Hakki et al., (Inter simple sequence repeats separate efficiently hemp from marijuana (Cannabis sativa L.), Electronic Journal of Biotechnology, 10(4), 2007); Gilmore et al. (Isolation of microsatellite markers in Cannabis sativa L. (marijuana), Molecular Ecology Notes, 3(1): 105-107, March 2003); Pacifico et al., (Genetics and marker assisted selection of chemotype in Cannabis sativa L.), Molecular Breeding (2006) 17:257-268); and Mendoza et al., (Genetic individualization of Cannabis sativa by a short tandem repeat multiplex system, Anal Bioanal Chem (2009) 393:719-726); each of which is herein incorporated by reference in its entirety. In some embodiments, the Cannabis plant, or plant part, tissue, or cell of ‘OT-1’ comprises a cannabinoid profile as set forth in Table 1 and a terpene profile as set forth in Table 2.

TABLE 1
Exemplary Profiles of Key Cannabinoids.

| | Percent | Percent | Percent |
|---|---|---|---|
| THCA | 11.456 | 14.32 | 17.184 |
| Total THC* | 10.312 | 12.89 | 15.468 |
| Total CBD | 0.04 | 0.05 | 0.06 |
| d9-THC | 0.264 | 0.33 | 0.396 |
| CBGA | 0.728 | 0.91 | 1.092 |
| Total | 12.528 | 15.66 | 18.792 |
| Cannabinoid | | | |

*Total THC = (THCA * 0.877) + THC (i.e. delta 9 THC) + delta 8 THC

TABLE 2
Exemplary Profiles of Key Terpenes.

| | Percent | Percent | Percent |
|---|---|---|---|
| Alpha Pinene | 0.0408 | 0.051 | 0.0612 |
| Fenchol | 0.0544 | 0.068 | 0.0816 |
| Terpineol | 0.044 | 0.055 | 0.066 |
| Alpha Humulene | 0.2016 | 0.252 | 0.3024 |
| (−)-Caryophyllene | 0.0184 | 0.023 | 0.0276 |
| Ocimene | 0.0336 | 0.042 | 0.0504 |
| Eucalyptol | 0.16 | 0.20 | 0.24 |
| Phytol | 0.0688 | 0.086 | 0.1032 |
| beta Myrcene | 0.0248 | 0.031 | 0.0372 |
| Limonene | 0.38 | 0.475 | 0.57 |
| Linalool | 0.0184 | 0.023 | 0.0276 |
| beta Caryophyllene | 0.392 | 0.490 | 0.588 |
| (−)-alpha Bisabolol | 0.0536 | 0.067 | 0.0804 |
| beta Pinene | 0.06 | 0.075 | 0.09 |

In some embodiments, there is about a 70:1 ratio of THC:CBD. In some embodiments, there is about 0.03-5% each of CBDV, CBC, CBCV, and THCV. In some embodiments, there is about 20% THC. In some embodiments, there is at least 1% CBG.

In some embodiments, the invention relates to a Cannabis clone regenerated from the Cannabis plant of descended from the plant, or plant part, tissue, cell or seed of ‘OT-1’ wherein the plant is a clonal descendent.

In some embodiments, the invention relates to a method of producing an F1 Cannabis seed, wherein the method includes crossing the plant with a different Cannabis plant and harvesting the resultant F1 Cannabis seed. In some embodiments, the invention relates to the F1 hybrid Cannabis seed produced by this method. In some embodiments, the invention relates to a F1 hybrid Cannabis plant produced by growing the F1 hybrid Cannabis seed. In some embodiments, the invention relates to a Cannabis clone regenerated from the F1 hybrid Cannabis plant. In some embodiments, the invention relates to a smokable or edible product comprising Cannabis tissue from the F1 hybrid Cannabis plant.

### Detailed Botanical Description

The following detailed description sets forth the distinctive characteristics of ‘OT-1’. Applicant is prepared to make a deposit of seeds or plant tissue in the event that claims are submitted reciting such a deposit of seeds or plant tissue.

Type: Herbaceous Tap-Rooted Annual

Classification: Strains of ‘Cannabis sativa’, possessing traits of the subspecies, ‘C. sativa ssp indica (Lamarck)’. When navigating the key of Small and Cronquist, ID., the first couplet separates individuals based on their ability to intoxicate. This cultivated line possesses intoxicating properties, and so the subspecies sativa and its varieties (var. sativa and spontanea) are eliminated from consideration. Within the next couplet distinguishing within the subspecies indica, fruits are required to separate between the varieties (var. indica and var kafiristanica). No fruits were found on any of the individuals observed, and so discrimination between the varieties is impossible with this key.
a. Family—Cannabaceae.
b. Genus—Cannabis.
c. Species—sativa.
d. Common Name—Marijuana/.

Market Class: A medicinal herb intended for use as medical oil, and medicinal herb for sale in cannabis dispensaries and as a home garden plant.

### Note

Applicant is prepared to submit a seed and/or tissue deposit of the cultivar herein described, prior to issuance or publication, as required by the law of the relevant jurisdiction, as needed to support claims reciting such a deposit.

1. A seed from Cannabis plant designated ‘OT-1’ wherein a representative sample of seed of said plant has been deposited under ______.

2. A Cannabis plant, or plant part, tissue, or cell thereof produced by growing the seed of claim 2, or a descendant thereof.

3. The Cannabis plant, or plant part, tissue, or cell thereof of claim 3 comprising a cannabinoid profile set forth in Table 1.

4. Use of the plant of claim 3 in a breeding program to produce cannabis progeny comprising a cannabinoid profile set forth in Table 1 and genetic capacity to produce the cannabinoid profile set forth in Table 1 in progeny thereof.

5. The Cannabis plant part of claim 3, wherein said plant part is selected from the group consisting of: stems, trichomes, leaves, and flower buds.

6. The Cannabis plant descended from the plant, or plant part, tissue, cell or seed of claim 3, wherein the plant is a clonal descendent.
